(19) United States
(12) Patent Application Publication
(10) Pub. No.: US 2022/0148721 A1
(43) Pub. Date: May 12, 2022

(54) ANALYSIS METHOD FOR CAUSAL INFERENCE OF PHYSIOLOGICAL NETWORK IN MULTISCALE TIME SERIES SIGNALS

(71) Applicant: University of Electronic Science and Technology of China, Chengdu (CN)

(72) Inventors: Yi Zhang, Chengdu (CN); Lifu Zhang, Chengdu (CN); Guan Wang, Chengdu (CN); Mingjun Xie, Chengdu (CN); Ziwen Li, Chengdu (CN); Denan Lin, Shenzhen (CN); Xiaohong Fan, Chengdu (CN); Banghua Wu, Chengdu (CN); Jipeng Fan, Chengdu (CN); Lixin Pu, Chengdu (CN); Mingjie He, Chengdu (CN); Steven Su, Sydney (AU); Branko Celler, Sydney (AU); Peng Xu, Chengdu (CN); Dezhong Yao, Chengdu (CN)

(73) Assignee: University of Electronic Science and Technology of China, Chengdu (CN)

(21) Appl. No.: 17/396,800

(22) Filed: Aug. 9, 2021

(30) Foreign Application Priority Data
Nov. 9, 2020 (CN) 202011239121.3

(51) Int. Cl. G16H 40/63; A61B 5/00; G06N 5/04

(52) U.S. Cl. CPC G16H 40/63; G06N 5/04; A61B 5/7235

(57) ABSTRACT

An analysis method for the causal inference of human physiological network in multiscale time series signals includes the following steps: S1: decomposing physiological signals u₁, u₂, . . . , uₘ to be analyzed by using a noise-assisted multivariate empirical mode decomposition (NA-MEND) algorithm; S2: carrying out a causal analysis between two different physiological signals uᵢ, uⱼ, where i=1, 2, . . . , m, j=1, 2, . . . , m, and i≠j, to obtain a causality between the two signals; and S3: repeating step S2 for any two signals in u₁, u₂, . . . , uₘ until a causality between each two signals in u₁, u₂, . . . , uₘ is obtained to form the causal network. The present invention can effectively analyze the causal network of the physiological signals, thereby facilitating the application of the physiological signals.

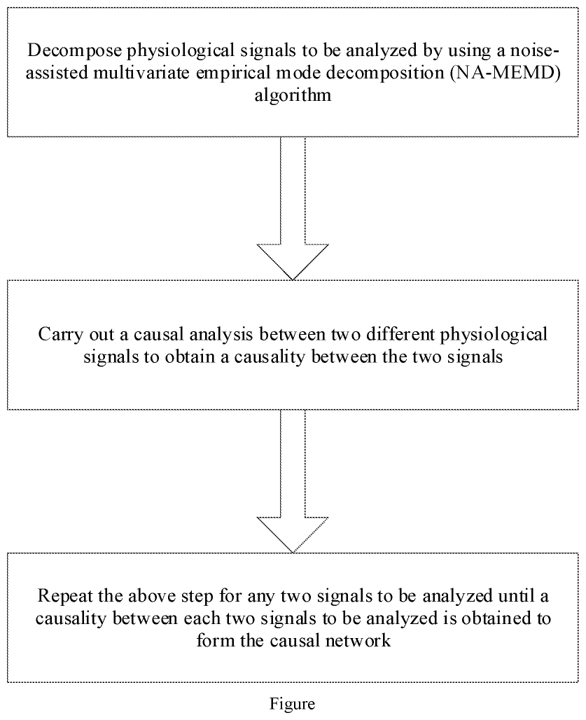

### CROSS REFERENCE TO THE RELATED APPLICATIONS

This application is based upon and claims priority to Chinese Patent Application No. 202011239121.3, filed on Nov. 9, 2020, the entire contents of which are incorporated herein by reference.

### TECHNICAL FIELD

The present invention relates to physiological signal processing, and in particular to an analysis method for the causal inference of human physiological network in multiscale time series signals.

### BACKGROUND

Analysis methods for the causal inference of the physiological network in multiscale time series signals can be applied to brain-computer interface (BCI) technology, brain function-structure mechanism research, and brain-apparatus (e.g., brains, eyes, hearts, lungs and muscles) conversation omics technology.

Since functional segregation and functional integration in neuroscience presented by Gall et al. show that distinct brain functions are localized in specialized cortical areas, the scientific criteria for assessing brain perception, cognition and behavior have been dominated by Granger causality analyses via functional connectivity (FC).

Cause and effect relationships in most real-world situations are likely time-dependent, simultaneous and reciprocal. However, existing methods for causality analyses in time series are mostly based on statistics and prediction, and may fail to describe a reciprocal causation motion between instantaneous events. In this context, the Granger causality is based on the assumption that the cause and effect are separable, which is useful in many linear stochastic systems, but might not be applicable in complex dynamical processes (e.g., brain-related physiological networks). Moreover, the convergent cross-mapping (CCM) method has been developed to accommodate the inseparability of causal effects. In the causal modeling of brain networks, dynamic causal modeling and transfer entropy are also prevalent but essentially based on Bayesian predictions. Yang et al. suggested an approach of assessing the causal interaction via Hilbert-Huang Transform (HHT). However, it may be incompatible with brain-linked complex information systems and also lack the necessary practices in priori cause-effect interactions.

The causal induction by Galilei and Hume underlying the covariation approach is that a cause-effect relationship is encoded from the sensory input in certain ways: that is, the covariation between a candidate cause and the effect can be defined as the difference between the probability of the effect given the presence of the candidate cause, and that probability given the absence of the candidate cause. Causal strength by the Kantian approach presents the existence of a priori knowledge to interpret the causal information. Compared with the generic HHT-based causal decomposition method, empirical mode decomposition (EMD) cannot guarantee the equal number of intrinsic mode functions (IMF s) across multiple time series, and lead to the mode (scale) alignment (cross-channel interdependence), which indicates the extent of time scales in multiple time series. For instance, in brain-physiological networks, oscillatory components observed from monitoring devices are often sampled in distinct frequency bands. It is also vulnerable to noise that may cause the mode mixing problem (single-channel independence), which remains insufficient to identify the decomposed intrinsic causal components (ICCs).

Until now, causality analysis over multiple time scales has been essentially applied to evaluate ICCs of time series. The multiscale Granger causality is solved by a filtering and down-sampling step to obtain the rescale representations of a bivariate process. The transfer entropy based multiscale analyses are mostly based on Fourier and wavelet transforms with the fixed basis functions. However, when applied to the complex process naturally inherited with nonlinear and non-stationary properties, those decomposition procedures do not capture the significant features across different time scales.

### SUMMARY

In order to overcome the shortcomings of the prior art, an objective of the present invention is to provide an analysis method for the causal inference of the physiological network in multiscale time series signals. The present invention can effectively analyze a causal relationship of the physiological network, thereby facilitating the application of the physiological signals.

The objective of the present invention is achieved by the following technical solution. An analysis method for the causal inference of human physiological network in multiscale time series signals includes the following steps:

S1: inputting physiological signals to be analyzed:

u₁={u_{1,1},u_{1,2}, . . . ,u_{1,t}}

u₂={u_{2,1},u_{2,2}, . . . ,u_{2,t}}

. . .

uₘ={u_{m,1},u_{m,2}, . . . ,u_{m,t}}

decomposing the physiological signals u₁, u₂, . . . , uₘ to be analyzed by using a noise-assisted multivariate empirical mode decomposition (NA-MEMD) algorithm:

u₁⇒{IMF_{1,1},IMF_{1,2}, . . . ,IMF_{1,n}}

u₂⇒{IMF_{2,1},IMF_{2,2}, . . . ,IMF_{2,n}}

. . .

uₘ⇒{IMF_{m,1},IMF_{m,2}, . . . ,IMF_{m,n}}

g₁⇒{IMF_{g}_{₁}_{,1},IMF_{g}_{₁}_{,2}, . . . ,IMF_{g}_{₁}_{,n}}

g₂⇒{IMF_{g}_{₂}_{,1},IMF_{g}_{₂}_{,2}, . . . ,IMF_{g}_{₂}_{,n}}

. . .

g_{\tilde{m}}⇒{IMF_{g}_{_{\tilde{m}}}_{,1},IMF_{g}_{_{\tilde{m}}}_{,2}, . . . ,IMF_{g}_{_{\tilde{m}}}_{,n}};

where, “⇒” represents the decomposition of the signal by the NA-MEMD algorithm; m represents the number of the physiological signals, m≥2, t∈N⁺, where N⁺ represents a positive integer; g₁, g₂, . . . , g_{\tilde{m}} represent assistant noises selected by the NA-MEMD algorithm, and g₁, g₂, . . . , g_{\tilde{m}} are uncorrelated random Gaussian noises; \tilde{m} represents the number of the assistant noises selected; n represents the number of intrinsic mode functions (IMFs) obtained after the decomposition of each of the physiological signals;

S2: carrying out a causal analysis between a physiological signal uᵢ and a physiological signal uⱼ, where i=1, 2, . . . , m, j=1, 2, . . . , m, and i≠j:

S201: pairing the IMFs {IMF_{i,1}, IMF_{i,2}, . . . , IMF_{i,n}} obtained by decomposing the physiological signal uᵢ with the IMFs {IMF_{j,1}, IMF_{j,2}, . . . , IMF_{j,n}} obtained by decomposing the physiological signal uⱼ to obtain n IMF pairs:
(IMF_{i,1},IMF_{j,1}), (IMF_{i,2},IMF_{j,2}), . . . , (IMF_{i,n},IMF_{j,n});

where, the two IMFs in each IMF pair of the n IMF pairs have the same length of time;

S202: calculating a mean instantaneous phase difference of the each IMF pair, comparing the mean instantaneous phase difference with a preset threshold to select IMF pairs each with a mean instantaneous phase difference less than the preset threshold, to generate intrinsic causal component (ICC) sets:
{(IMF_{i,k}_{₁},IMF_{j,k}_{₁}), (IMF_{i,k}_{₂},IMF_{j,k}_{₂}), . . . , (IMF_{i,}_{ₙ},IMF_{j,}_{ₙ})};

where, k₁ in IMF_{i,k}_{₁} represents that IMF_{i,k}_{₁} is a k₁-th IMF in {IMF_{i,1}, IMF_{i,2}, . . . , IMF_{i,n}}, and k₁ in IMF_{j,k}_{₁} represents that IMF_{j,k}_{₁} is a k₁-th IMF in {IMF_{j,1}, IMF_{j,2}, . . . , IMF_{j,n}};

k₂ in IMF_{i,k}_{₂} represents that IMF_{i,k}_{₂} is a k₂-th IMF in {IMF_{i,1}, IMF_{i,2}, . . . , IMF_{i,n}}, and k₂ in IMF_{j,k}_{₂} represents that IMF_{j,k}_{₂} is a k₂-th IMF in {IMF_{j,1}, IMF_{j,2}, . . . , IMF_{j,n}};

similarly, k_{ñ} in IMF_{i,k}_{_{ñ}} represents that IMF_{i,k}_{_{ñ}} is a k_{ñ}-th IMF in {IMF_{i,1}, IMF_{i,2}, . . . , IMF_{i,n}}, and k_{ñ} in IMF_{j,k}_{_{ñ}} represents that IMF_{j,k}_{_{ñ}} is a k_{ñ}-th IMF in {IMF_{j,1}, IMF_{j,2}, . . . , IMF_{j,n}};

ñ represents the number of the IMF pairs in the ICC sets;

S203: calculating a phase coherence of each of the IMF pairs in the ICC sets respectively:

$$Coh ( {IMF}_{i , k} \to {IMF}_{j , k} ) = \frac{1}{T} \vert {\int }_{0}^{T} {e}^{i [ {ϕ}_{i , k} ( t ) - {ϕ}_{j , k} ( t ) ]} dt \vert ;$$

where, k=k₁, k₂, . . . , k_{ñ}; T represents the length of time of IMF_{i,k} and IMF_{j,k}; ϕ_{i,k}(t) represents an instantaneous phase of IMF_{i,k} at a time t, and ϕ_{j,k}(t) represents an instantaneous phase of IMF_{j,k} at the time t;

S204: signal re-decomposition:

selecting an IMF pair with a highest frequency from the IMF pairs corresponding to serial numbers in the ICC sets, where since the frequencies of the IMFs decomposed by the NA-MEND algorithm are arranged in descending order, the IMF pair with the highest frequency is (IMF_{i,k}_{₁},IMF_{j,k}_{₁});

subtracting IMF_{j,k}_{₁} from the physiological signal uⱼ to obtain uⱼ′, replacing uⱼ in an input signal set u₁, u₂, . . . , uₘ with uⱼ′ to obtain a first replaced input signal set, and carrying out a first NA-MEMD decomposition on the first replaced input signal set;

obtaining decomposed IMFs {IMF_{j,1}′, IMF_{j,2}′, . . . , IMF_{j,n}′} corresponding to uⱼ′ after the first NA-MEMD decomposition;

subtracting IMF_{i,k}_{₁} from the physiological signal uᵢ to obtain uᵢ′, replacing uᵢ in an input signal set u₁, u₂, . . . , uₘ with uᵢ′ to obtain a second replaced input signal set, and carrying out a second NA-MEMD decomposition on the second replaced input signal set;

obtaining decomposed IMFs {IMF_{i,1}′, IMF_{i,2}′, . . . , IMF_{i,n}′} corresponding to uᵢ′ after the second NA-MEMD decomposition;

S205: calculating a causality D(IMF_{i,k}_{₁}→IMF_{j,k}_{₁}) of uᵢ to uⱼ and a causality D(IMF_{j,k}_{₁}→IMF_{i,k}_{₁}) of uⱼ to uᵢ:

$$\{ \begin{matrix}D ( {IMF}_{i , {k}_{1}} \to {IMF}_{j , {k}_{1}} ) = {\{ {\sum }_{k = {k}_{1}}^{{k}_{\tilde{n}}} {{W}_{k} [ Coh ( {IMF}_{i , k} \to {IMF}_{j , k} ) - Coh ( {IMF}_{i , k} \to {IMF}_{j , k}^{′} ) ]}^{2} \}}^{\frac{1}{2}} \\ D ( {IMF}_{j , {k}_{1}} \to {IMF}_{i , {k}_{1}} ) = {\{ {\sum }_{k = {k}_{1}}^{{k}_{\tilde{n}}} {{W}_{k} [ Coh ( {IMF}_{i , k} \to {IMF}_{j , k} ) - Coh ( {IMF}_{i , k}^{′} \to {IMF}_{j , k} ) ]}^{2} \}}^{\frac{1}{2}} \\ {W}_{k} = ( {σ}_{i , k}^{2} \times {σ}_{j , k}^{2} ) / {\sum }_{k = {k}_{1}}^{{k}_{\tilde{n}}} ( {σ}_{i , k}^{2} \times {σ}_{j , k}^{2} )\end{matrix} ;$$

where, σ_{i,k}² is a variance of a k-th IMF obtained by decomposing uᵢ, and σ_{j,k}² is a variance of a k-th IMF obtained by decomposing uⱼ; wₖ is an intermediate variable;

obtaining an absolute causal strength (ACS):

ACS={D(IMF_{i,k}_{₁}→IMF_{j,k}_{₁}),D(IMF_{j,k}_{₁}→IMF_{i,k}_{₁})};

S206: based on the ACS, calculating a ratio:

$$\frac{D ( {IMF}_{i , {k}_{1}} \to {IMF}_{j , {k}_{1}} )}{D ( {IMF}_{j , {k}_{1}} \to {IMF}_{i , {k}_{1}} )} ;$$

where, if the ratio is greater than 1, then uᵢ is a cause and uⱼ is an effect;

if the ratio is less than 1, then uᵢ is the effect and uⱼ is the cause;

if the ratio is equal to 1, then uᵢ and uⱼ are reciprocal causation or are not causation;

in this way, causal analysis results of uᵢ and uⱼ are obtained;

S3: repeating step S2 for any two signals in u₁, u₂, . . . , uₘ until a causality between each two signals in u₁, u₂, . . . , uₘ is obtained to form the causal network.

Step S202 includes:

S2021: setting mean instantaneous phase difference thresholds δ₁, δ₂, . . . , δₙ for the n IMF pairs;

S2022: calculating a mean instantaneous phase difference of an h-th IMF pair (IMF_{i,h},IMF_{j,h});

letting mean(ϕ_{i,h}) be a mean instantaneous phase of IMF_{i,h} in the length of time, and letting mean(ϕ_{j,h}) be a mean instantaneous phase of IMF_{j,h} in the length of time;

then obtaining the mean instantaneous phase difference of the h-th IMF pair (IMF_{i,h},IMF_{j,h}) as:

|mean(ϕ_{i,h})−mean(ϕ_{j,h})|;

comparing |mean(ϕ_{i,h})−mean(ϕ_{j,h})| with a corresponding threshold δₕ, and determining whether the following condition is satisfied:

|mean(ϕ_{i,h})−mean(ϕ_{j,h})|<δₕ;

if the condition is satisfied, then adding the h-th IMF pair (IMF_{i,h},IMF_{j,h}) into the ICC sets;

if the condition is not satisfied, then discarding (IMF_{i,h},IMF_{j,h});

S2023: repeating step S2022 when h=1, 2, . . . n respectively to finally obtain the ICC sets as:
{(IMF_{i,k}_{₁},IMF_{j,k}_{₁}), (IMF_{i,k}_{₂},IMF_{j,k}_{₂}), . . . , (IMF_{i,}_{ₙ},IMF_{j,}_{ₙ})}.

The present invention has the following advantages. The analysis method for the causal inference of the physiological network in multiscale time series signals provided by the present invention effectively analyzes the causal network of the physiological signals. Compared with traditional methods, the analysis results of the present invention can represent the causality in the time series more completely, thereby providing technical conditions for the application of the physiological signals in brain-computer interface (BCI) technology, brain function-structure mechanism research and brain-apparatus conversation omics technology.

### BRIEF DESCRIPTION OF THE DRAWINGS

FIGURE is a flowchart of the method according to the present invention.

### DETAILED DESCRIPTION OF THE EMBODIMENTS

The technical solutions of the present invention are described in further detail below with reference to the drawings, but the scope of protection of the present invention is not limited thereto.

As shown in FIGURE, an analysis method for the causal inference of human physiological network in multiscale time series signals includes the following steps:

S1: physiological signals to be analyzed are input:

u₁={u_{1,1},u_{1,2}, . . . ,u_{1,t}}

u₂={u_{2,1},u_{2,2}, . . . ,u_{2,t}}

. . .

uₘ={u_{m,1},u_{m,2}, . . . ,u_{m,t}};

the physiological signals u₁, u₂, . . . , uₘ to be analyzed are decomposed by using a noise-assisted multivariate empirical mode decomposition (NA-MEMD) algorithm:

u₁⇒{IMF_{1,1},IMF_{1,2}, . . . ,IMF_{1,n}}

u₂⇒{IMF_{2,1},IMF_{2,2}, . . . ,IMF_{2,n}}

. . .

uₘ⇒{IMF_{m,1},IMF_{m,2}, . . . ,IMF_{m,n}}

g₁⇒{IMF_{g}_{₁}_{,1},IMF_{g}_{₁}_{,2}, . . . ,IMF_{g}_{₁}_{,n}}

g₂⇒{IMF_{g}_{₂}_{,1},IMF_{g}_{₂}_{,2}, . . . ,IMF_{g}_{₂}_{,n}}

. . .

g_{\tilde{m}}⇒{IMF_{g}_{_{\tilde{m}}}_{,1},IMF_{g}_{_{\tilde{m}}}_{,2}, . . . ,IMF_{g}_{_{\tilde{m}}}_{,n}};

where, “⇒” represents the decomposition of the signal by the NA-MEMD algorithm; m represents the number of the physiological signals, m≥2, t∈N⁺, where N⁺ represents a positive integer; g₁, g₂, . . . , g_{\tilde{m}} represent assistant noises selected by the NA-MEMD algorithm, and g₁, g₂, . . . , g_{\tilde{m}} are uncorrelated random Gaussian noises; \tilde{m} represents the number of the assistant noises selected; n represents the number of intrinsic mode functions (IMFs) obtained after the decomposition of each of the physiological signals.

S2: a causal analysis is carried out between a physiological signal uᵢ and a physiological signal uⱼ, where i=1, 2, . . . , m, j=1, 2, . . . , m, and i≠j:

S201: the IMFs {IMF_{i,1}, IMF_{i,2}, . . . , IMF_{i,n}} obtained by decomposing the physiological signal uᵢ is paired with the IMFs {IMF_{j,1}, IMF_{j,2}, . . . , IMF_{j,n}} obtained by decomposing the physiological signal uⱼ to obtain n IMF pairs:
(IMF_{i,1},IMF_{j,1}), (IMF_{i,2},IMF_{j,2}), . . . , (IMF_{i,n},IMF_{j,n});

where, the two IMFs in each IMF pair of the n IMF pairs have the same length of time.

S202: a mean instantaneous phase difference of the each IMF pair is calculated, the mean instantaneous phase difference is compared with a preset threshold to select IMF pairs each with a mean instantaneous phase difference less than the preset threshold, to generate intrinsic causal component (ICC) sets:
{(IMF_{i,k}_{₁},IMF_{j,k}_{₁}), (IMF_{i,k}_{₂},IMF_{j,k}_{₂}), . . . , (IMF_{i,}_{ₙ},IMF_{j,}_{ₙ})};

where, k₁ in IMF_{i,k}_{₁} represents that IMF_{i,k}_{₁} is a k₁-th IMF in {IMF_{i,1}, IMF_{i,2}, . . . , IMF_{i,n}}, and k₁ in IMF_{j,k}_{₁} represents that IMF_{j,k}_{₁} is a k₁-th IMF in {IMF_{j,1}, IMF_{j,2}, . . . , IMF_{j,n}};

k₂ in IMF_{i,k}_{₂} represents that IMF_{i,k}_{₂} is a k₂-th IMF in {IMF_{i,1}, IMF_{i,2}, . . . , IMF_{i,n}}, and k₂ in IMF_{j,k}_{₂} represents that IMF_{j,k}_{₂} is a k₂-th IMF in {IMF_{j,1}, IMF_{j,2}, . . . , IMF_{j,n}};

similarly, k_{ñ} in IMF_{i,k}_{_{ñ}} represents that IMF_{i,k}_{_{ñ}} is a k_{ñ}-th IMF in {IMF_{i,1}, IMF_{i,2}, . . . , IMF_{i,n}}, and k_{ñ} in IMF_{j,k}_{_{ñ}} represents that IMF_{j,k}_{_{ñ}} is a k_{ñ}-th IMF in {IMF_{j,1}, IMF_{j,2}, . . . , IMF_{j,n}}; and

ñ represents the number of the IMF pairs in the ICC sets.

S203: a phase coherence of each of the IMF pairs in the ICC sets is calculated respectively:

$$Coh ( {IMF}_{i , k} \to {IMF}_{j , k} ) = \frac{1}{T} \vert {\int }_{0}^{T} {e}^{i [ {ϕ}_{i , k} ( t ) - {ϕ}_{j , k} ( t ) ]} dt \vert ;$$

where, k=k₁, k₂, . . . , k_{ñ}; T represents the length of time of IMF_{i,k} and IMF_{j,k}; ϕ_{i,k}(t) represents an instantaneous phase of IMF_{i,k} at a time t, and ϕ_{j,k}(t) represents an instantaneous phase of IMF_{j,k} at the time t.

S204: signal re-decomposition:

an IMF pair with a highest frequency is selected from the IMF pairs corresponding to serial numbers in the ICC sets, where since the frequencies of the IMFs decomposed by the NA-MEND algorithm are arranged in descending order, the IMF pair with the highest frequency is (IMF_{i,k}_{₁},IMF_{j,k}_{₁});

IMF_{j,k}_{₁} is subtracted from the physiological signal uⱼ to obtain uⱼ′, uⱼ in an input signal set u₁, u₂, . . . , uₘ is replaced with uⱼ′ to obtain a first replaced input signal set, and a first NA-MEMD decomposition is carried out on the first replaced input signal set;

decomposed IMFs {IMF_{j,1}′, IMF_{j,2}′, . . . , IMF_{j,n}′} corresponding to uⱼ′ are obtained after the first NA-MEMD decomposition;

IMF_{i,k}_{₁} is subtracted from the physiological signal uᵢ to obtain uᵢ′, uᵢ in an input signal set u₁, u₂, . . . , uₘ is replaced with uᵢ′ to obtain a second replaced input signal set, and a second NA-MEMD decomposition is carried out on the second replaced input signal set;

decomposed IMFs {IMF_{i,1}′, IMF_{i,2}′, . . . , IMF_{i,n}′} corresponding to uᵢ′ are obtained after the second NA-MEMD decomposition;

S205: a causality D(IMF_{i,k}_{₁}→IMF_{j,k}_{₁}) of uᵢ to uⱼ and a causality D(IMF_{j,k}_{₁}→IMF_{i,k}_{₁}) of uⱼ to uᵢ are calculated:

$$\{ \begin{matrix}D ( {IMF}_{i , {k}_{1}} \to {IMF}_{j , {k}_{1}} ) = {\{ {\sum }_{k = {k}_{1}}^{{k}_{\tilde{n}}} {{W}_{k} [ Coh ( {IMF}_{i , k} \to {IMF}_{j , k} ) - Coh ( {IMF}_{i , k} \to {IMF}_{j , k}^{′} ) ]}^{2} \}}^{\frac{1}{2}} \\ D ( {IMF}_{j , {k}_{1}} \to {IMF}_{i , {k}_{1}} ) = {\{ {\sum }_{k = {k}_{1}}^{{k}_{\tilde{n}}} {{W}_{k} [ Coh ( {IMF}_{i , k} \to {IMF}_{j , k} ) - Coh ( {IMF}_{i , k}^{′} \to {IMF}_{j , k} ) ]}^{2} \}}^{\frac{1}{2}} \\ {W}_{k} = ( {σ}_{i , k}^{2} \times {σ}_{j , k}^{2} ) / {\sum }_{k = {k}_{1}}^{{k}_{\tilde{n}}} ( {σ}_{i , k}^{2} \times {σ}_{j , k}^{2} )\end{matrix} ;$$

where, σ_{i,k}² is a variance of a k-th IMF obtained by decomposing uᵢ, and σ_{j,k}² is a variance of a k-th IMF obtained by decomposing uⱼ; wₖ is an intermediate variable; and

an absolute causal strength (ACS) is obtained:

ACS={D(IMF_{i,k}_{₁}→IMF_{j,k}_{₁}),D(IMF_{j,k}_{₁}→IMF_{i,k}_{₁})}.

S206: based on the ACS, a ratio is calculated:

$$\frac{D ( {IMF}_{i , {k}_{1}} \to {IMF}_{j , {k}_{1}} )}{D ( {IMF}_{j , {k}_{1}} \to {IMF}_{i , {k}_{1}} )} ;$$

where, if the ratio is greater than 1, then uᵢ is a cause and uⱼ is an effect;

if the ratio is less than 1, then uᵢ is the effect and uⱼ is the cause;

if the ratio is equal to 1, then uᵢ and uⱼ are reciprocal causation or are not causation; and

in this way, causal analysis results of uᵢ and uⱼ are obtained.

S3: step S2 is repeated for any two signals in u₁, u₂, . . . , uₘ until a causality between each two signals in u₁, u₂, . . . , uₘ is obtained to form the causal network.

Step S202 includes:

S2021: mean instantaneous phase difference thresholds δ₁, δ₂, . . . , δₙ for the n IMF pairs are set.

S2022: a mean instantaneous phase difference of an h-th IMF pair (IMF_{i,h},IMF_{j,h}) is calculated as follows:

let mean(ϕ_{i,h}) be a mean instantaneous phase of IMF_{i,h} in the length of time, and let mean(ϕ_{j,h}) be a mean instantaneous phase of IMF_{j,h} in the length of time;

then the mean instantaneous phase difference of the h-th IMF pair (IMF_{i,h},IMF_{j,h}) is obtained as:

|mean(ϕ_{i,h})−mean(ϕ_{j,h})|;

|mean(ϕ_{i,h})−mean(ϕ_{j,h})| is compared with the corresponding threshold δₕ, and it is determined whether the following condition is satisfied:

|mean(ϕ_{i,h})−mean(ϕ_{j,h})|<δₕ;

if the condition is satisfied, then the h-th IMF pair (IMF_{i,h},IMF_{j,h}) is added into the ICC sets; and

if the condition is not satisfied, then (IMF_{i,h},IMF_{j,h}) is discarded.

S2023: step S2022 is repeated when h=1, 2, . . . n respectively to finally obtain the ICC sets as:
{(IMF_{i,k}_{₁},IMF_{j,k}_{₁}), (IMF_{i,k}_{₂},IMF_{j,k}_{₂}), . . . , (IMF_{i,}_{ₙ},IMF_{j,}_{ₙ})}.

In the embodiment of the present invention, the causality between two physiological signals A and B can be defined as follows: if the decomposed IMFs in both A and B are at a certain similar time scale and the IMF in B is removed from B itself, variable A causes variable B if the instantaneous phase dependency between the IMFs in A and B is eliminated, but not vice versa, namely variable A does not cause variable B if the instantaneous phase dependency between the IMFs in A and B is not eliminated.

The causality needs to satisfy the following conditions:

(1) any causality is based on the instantaneous phase coherence of the ICCs across multiple time series; and

(2) the phase behaviors in an effect are separable from the effect itself.

The specific implementations of the present invention are described above, but those skilled in the art should understand that they are only illustrative, and various changes or modifications may be made to these implementations without departing from the principle and implementation of the present invention. Therefore, the scope of protection of the present invention is defined by the appended claims.

What is claimed is:

1. An analysis method for a causal inference of a physiological network in multiscale time series signals, comprising the following steps:
S1: inputting physiological signals to be analyzed:
u₁={u_{1,1},u_{1,2}, . . . ,u_{1,t}}
u₂={u_{2,1},u_{2,2}, . . . ,u_{2,t}}
. . .
uₘ={u_{m,1},u_{m,2}, . . . ,u_{m,t}};
decomposing the physiological signals u₁, u₂, . . . , uₘ to be analyzed by using a noise-assisted multivariate empirical mode decomposition (NA-MEMD) algorithm:
u₁⇒{IMF_{1,1},IMF_{1,2}, . . . ,IMF_{1,n}}
u₂⇒{IMF_{2,1},IMF_{2,2}, . . . ,IMF_{2,n}}
. . .
uₘ⇒{IMF_{m,1},IMF_{m,2}, . . . ,IMF_{m,n}}
g₁⇒{IMF_{g}_{₁}_{,1},IMF_{g}_{₁}_{,2}, . . . ,IMF_{g}_{₁}_{,n}}
g₂⇒{IMF_{g}_{₂}_{,1},IMF_{g}_{₂}_{,2}, . . . ,IMF_{g}_{₂}_{,n}}
. . .
g_{\tilde{m}}⇒{IMF_{g}_{_{\tilde{m}}}_{,1},IMF_{g}_{_{\tilde{m}}}_{,2}, . . . ,IMF_{g}_{_{\tilde{m}}}_{,n}};
wherein, “⇒” represents a decomposition of a signal by the NA-MEMD algorithm; m represents a number of the physiological signals, m≥2, t∈N⁺, wherein N⁺ represents a positive integer; g₁, g₂, . . . , g_{\tilde{m}} represent assistant noises selected by the NA-MEMD algorithm, and g₁, g₂, . . . , g_{\tilde{m}} are uncorrelated random Gaussian noises; \tilde{m} represents a number of the assistant noises selected; n represents a number of intrinsic mode functions (IMFs) obtained after a decomposition of each of the physiological signals;
S2: carrying out a causal analysis between a physiological signal uᵢ and a physiological signal uⱼ, where i=1, 2, . . . , m, j=1, 2, . . . , m, and i≠j:
S201: pairing IMFs {IMF_{i,1}, IMF_{i,2}, . . . , IMF_{i,n}} obtained by decomposing the physiological signal uᵢ with the IMFs {IMF_{j,1}, IMF_{j,2}, . . . , IMF_{j,n}} obtained by decomposing the physiological signal uⱼ to obtain n IMF pairs:
(IMF_{i,1},IMF_{j,1}), (IMF_{i,2},IMF_{j,2}), . . . , (IMF_{i,n},IMF_{j,n});
where, the two IMFs in each IMF pair of the n IMF pairs have the same length of time;
S202: calculating a mean instantaneous phase difference of the each IMF pair, comparing the mean instantaneous phase difference with a preset threshold to select IMF pairs each with a mean instantaneous phase difference less than the preset threshold, to generate intrinsic causal component (ICC) sets:
{(IMF_{i,k}_{₁},IMF_{j,k}_{₁}), (IMF_{i,k}_{₂},IMF_{j,k}_{₂}), . . . , (IMF_{i,}_{ₙ},IMF_{j,}_{ₙ})};
where, k₁ in IMF_{i,k}_{₁} represents that IMF_{i,k}_{₁} is a k₁-th IMF in {IMF_{i,1}, IMF_{i,2}, . . . , IMF_{i,n}}, and k₁ in IMF_{j,k}_{₁} represents that IMF_{j,k}_{₁} is a k₁-th IMF in {IMF_{j,1}, IMF_{j,2}, . . . , IMF_{j,n}};
k₂ in IMF_{i,k}_{₂} represents that IMF_{i,k}_{₂} is a k₂-th IMF in {IMF_{i,1}, IMF_{i,2}, . . . , IMF_{i,n}}, and k₂ in IMF_{j,k}_{₂} represents that IMF_{j,k}_{₂} is a k₂-th IMF in {IMF_{j,1}, IMF_{j,2}, . . . , IMF_{j,n}};
similarly, k_{ñ} in IMF_{i,k}_{_{ñ}} represents that IMF_{i,k}_{_{ñ}} is a k_{ñ}-th IMF in {IMF_{i,1}, IMF_{i,2}, . . . , IMF_{i,n}}, and k_{ñ} in IMF_{j,k}_{_{ñ}} represents that IMF_{j,k}_{_{ñ}} is a k_{ñ}-th IMF in {IMF_{j,1}, IMF_{j,2}, . . . , IMF_{j,n}};
ñ represents the number of the IMF pairs in the ICC sets;
S203: calculating a phase coherence of each of the IMF pairs in the ICC sets respectively:
$$Coh ( {IMF}_{i , k} \to {IMF}_{j , k} ) = \frac{1}{T} \vert {\int }_{0}^{T} {e}^{i [ {ϕ}_{i , k} ( t ) - {ϕ}_{j , k} ( t ) ]} dt \vert ;$$
where, k=k₁, k₂, . . . , k_{ñ}; T represents the length of time of IMF_{i,k} and IMF_{j,k}; ϕ_{i,k}(t) represents an instantaneous phase of IMF_{i,k} at a time t, and ϕ_{j,k}(t) represents an instantaneous phase of IMF_{j,k} at the time t;
S204: signal re-decomposition:
selecting an IMF pair with a highest frequency from the IMF pairs corresponding to serial numbers in the ICC sets, where since the frequencies of the IMFs decomposed by the NA-MEND algorithm are arranged in descending order, the IMF pair with the highest frequency is (IMF_{i,k}_{₁},IMF_{j,k}_{₁});
subtracting IMF_{j,k}_{₁} from the physiological signal uⱼ to obtain uⱼ′, replacing uⱼ in an input signal set u₁, u₂, . . . , uₘ with uⱼ′ to obtain a first replaced input signal set, and carrying out a first NA-MEMD decomposition on the first replaced input signal set;
obtaining decomposed IMFs {IMF_{j,1}′, IMF_{j,2}′, . . . , IMF_{j,n}′} corresponding to uⱼ′ after the first NA-MEMD decomposition;
subtracting IMF_{i,k}_{₁} from the physiological signal uᵢ to obtain uᵢ′, replacing uᵢ in an input signal set u₁, u₂, . . . , uₘ with uᵢ′ to obtain a second replaced input signal set, and carrying out a second NA-MEMD decomposition on the second replaced input signal set;
obtaining decomposed IMFs {IMF_{i,1}′, IMF_{i,2}′, . . . , IMF_{i,n}′} corresponding to uᵢ′ after the second NA-MEMD decomposition;
S205: calculating a causality D(IMF_{i,k}_{₁}→IMF_{j,k}_{₁}) of uᵢ to uⱼ and a causality D(IMF_{j,k}_{₁}→IMF_{i,k}_{₁}) of uⱼ to uᵢ:
$$\{ \begin{matrix}D ( {IMF}_{i , {k}_{1}} \to {IMF}_{j , {k}_{1}} ) = {\{ {\sum }_{k = {k}_{1}}^{{k}_{\tilde{n}}} {{W}_{k} [ Coh ( {IMF}_{i , k} \to {IMF}_{j , k} ) - Coh ( {IMF}_{i , k} \to {IMF}_{j , k}^{′} ) ]}^{2} \}}^{\frac{1}{2}} \\ D ( {IMF}_{j , {k}_{1}} \to {IMF}_{i , {k}_{1}} ) = {\{ {\sum }_{k = {k}_{1}}^{{k}_{\tilde{n}}} {{W}_{k} [ Coh ( {IMF}_{i , k} \to {IMF}_{j , k} ) - Coh ( {IMF}_{i , k}^{′} \to {IMF}_{j , k} ) ]}^{2} \}}^{\frac{1}{2}} \\ {W}_{k} = ( {σ}_{i , k}^{2} \times {σ}_{j , k}^{2} ) / {\sum }_{k = {k}_{1}}^{{k}_{\tilde{n}}} ( {σ}_{i , k}^{2} \times {σ}_{j , k}^{2} )\end{matrix} ;$$
wherein, σ_{i,k}² is a variance of a k-th IMF obtained by decomposing uᵢ, and σ_{j,k}² is a variance of a k-th IMF obtained by decomposing uⱼ; wₖ is an intermediate variable;
obtaining an absolute causal strength (ACS):
ACS={D(IMF_{i,k}_{₁}→IMF_{j,k}_{₁}),D(IMF_{j,k}_{₁}→IMF_{i,k}_{₁})};
S206: based on the ACS, calculating a ratio:
$$\frac{D ( {IMF}_{i , {k}_{1}} \to {IMF}_{j , {k}_{1}} )}{D ( {IMF}_{j , {k}_{1}} \to {IMF}_{i , {k}_{1}} )} ;$$
wherein, if the ratio is greater than 1, then uᵢ is a cause and uⱼ is an effect;
if the ratio is less than 1, then uᵢ is the effect and uⱼ is the cause;
if the ratio is equal to 1, then uᵢ and uⱼ are reciprocal causation or are not causation;
in this way, causal analysis results of uᵢ and uⱼ are obtained; and
S3: repeating step S2 for any two signals in u₁, u₂, . . . , uₘ until a causality between each two signals in u₁, u₂, . . . , uₘ is obtained to form the causal network.

2. The analysis method for the causal inference of the physiological network in the multiscale time series signals according to claim 1, wherein
step S202 comprises the following steps:
S2021: setting mean instantaneous phase difference thresholds δ₁, δ₂, . . . , δₙ for the n IMF pairs;
S2022: calculating a mean instantaneous phase difference of an h-th IMF pair (IMF_{i,h},IMF_{j,h});
letting mean(ϕ_{i,h}) be a mean instantaneous phase of IMF_{i,h} in the length of time, and letting mean(ϕ_{j,h}) be a mean instantaneous phase of IMF_{j,h} in the length of time;
then obtaining the mean instantaneous phase difference of the h-th IMF pair (IMF_{i,h},IMF_{j,h}) as:

|mean(ϕ_{i,h})−mean(ϕ_{j,h})|;
comparing |mean(ϕ_{i,h})−mean(ϕ_{j,h})| with a corresponding threshold δₕ, and determining whether the following condition is satisfied:

|mean(ϕ_{i,h})−mean(ϕ_{j,h})|<δₕ;
if the condition is satisfied, then adding the h-th IMF pair (IMF_{i,h},IMF_{j,h}) into the ICC sets;
if the condition is not satisfied, then discarding (IMF_{i,h},IMF_{j,h}); and
S2023: repeating step S2022 when h=1, 2, . . . n respectively to finally obtain the ICC sets as:
{(IMF_{i,k}_{₁},IMF_{j,k}_{₁}), (IMF_{i,k}_{₂},IMF_{j,k}_{₂}), . . . , (IMF_{i,}_{ₙ},IMF_{j,}_{ₙ})}.
